(19) United States
(12) Patent Application Publication
(10) Pub. No.: US 2003/0228287 A1
(43) Pub. Date: Dec. 11, 2003

(54) Maintenance of islet cells

(75) Inventors: Gillian M. Beattie, San Diego, CA (US); Alberto Hayek, La Jolla, CA (US); Anthony Montgomery, Cardiff, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California

(21) Appl. No.: 10/274,529

(22) Filed: Oct. 18, 2002

Related U.S. Application Data
(60) Provisional application No. 60/387,307, filed on Jun. 7, 2002.

(51) Int. Cl. A61K 45/00; C12N 5/08

(52) U.S. Cl. 424/93.7; 435/366

(57) ABSTRACT

The present invention provides a method of culturing cells on a matrix. The present invention includes islet cells cultured in a 3-D configuration in the presence of a fibrin matrix support. Induction of β-cell proliferation with HGF/SF is also provided.

### CROSS-REFERENCES TO RELATED APPLICATIONS

This application claims the benefit of U.S. Provisional Patent Application Serial No. 60/387,307, filed Jun. 7, 2002 which is herein incorporated by reference in its entirety for all purposes.

### BACKGROUND OF THE INVENTION

Type 1 (insulin-dependent) diabetes mellitus is characterized, inter alia, by a loss of insulin-producing β-cells and decompensation of metabolism following autoimmune aggression. See, e.g., Eisenbarth (1986) N. Eng. J. Med. 314:1360; Swenne (1992) Diabetologia 35:193. The loss of β cells impairs the body's ability to assimilate glucose from the blood, and the resulting high glucose levels can lead to blindness, kidney disease, nerve damage, and ultimately death. Insulin injections are commonly used to compensate for the lack of β cells, but blood sugar levels can still fluctuate widely. Methods of lessening the fluctuations have included the use of small, frequent doses of insulin and the use of mechanical pumps that mimic the action of the pancreas, but these require continuous or periodic maintenance, and the results are often of limited success. An alternative is a pancreatic transplant, but this requires major surgery and the availability of donor pancreases is limited.

A more promising option is the transplantation of islets of Langerhans. Recently, adult human pancreatic islets have transplanted into patients as an alternative to insulin injections. See, e.g., Scharp et al. (1991) Transplant. 51:76; Warnock et al. (1991) Diabetologia 34: 55.

One means of obtaining a sufficient number of islets for transplantation is ex vivo or in vitro proliferation. Successful ex vivo or in vitro proliferation occurs when the proliferated cells retain their functionality, i.e., their ability to produce insulin. However, appropriate regulation of the proliferation and differentiation of human fetal pancreatic islet cells has remained elusive.

Thus, there is a need in the art for new methods of culturing cells to maintain the differentiated state and protein expression of cells. In particular, there is a need for a new method of culturing islet cells that will result in proliferation at a substantial rate with minimal or no loss of insulin secreting function. The present invention addresses these and other problems.

### BRIEF SUMMARY OF THE INVENTION

The present invention provides a method of culturing cells on a matrix.

One embodiment of the present invention provides a method of culturing cells on a matrix comprising an integrin ligand. In some embodiments, the integrin ligand is an a5β1 integrin ligand, an αvβ1 integrin ligand, or combinations thereof. In other embodiments, the matrix further comprises a synthetic mesh. In even other embodiments, the integrin ligand is fibrin, fibronectin, or vitronectin. In yet other embodiments, the culturing step comprises mixing fibrinogen and thrombin, thereby forming a matrix comprising fibrin. In some embodiments, the cells are, for example, islet cells or human islet cells. In other embodiments, the method further comprises contacting the cells with hepatocyte growth factor, kaposis fibroblast growth factor, nicotinamide, or a combination thereof. In yet other embodiments, the hepatocyte growth factor is present at 10 ng/ml to 50 ng/ml. In even other embodiments, the kaposis fibroblast growth factor is present at 1 ng/ml to 50 ng/ml. In further embodiments, the nicotinamide is present at 1 mM to 50 mM. According to some embodiments, de-differentiation or senescence of the cells may be prevented.

Another embodiment of the present invention provides a method of cell transplantation. Cells are cultured on a matrix comprising an integrin ligand and administered to a mammal. The integrin ligand is an α5β1 integrin ligand, an αvβ1 integrin ligand, or combinations thereof. In some embodiments, administration is to a diabetic mammal. In other embodiments, the cells are islet cells. In even other embodiments, the matrix further comprises a synthetic mesh. In some embodiments, the integrin ligand is fibrin, fibronectin, vitronectin, or combinations thereof. In some embodiments, the culturing step comprises mixing fibrinogen and thrombin, thereby forming a matrix comprising fibrin. In yet other embodiments, the culturing step further comprises contacting the cells with hepatocyte growth factor, kaposis fibroblast growth factor and nicotinamide, or a combination thereof. In further embodiments, the hepatocyte growth factor is present at 10 ng/ml to 50 ng/ml. In even further embodiments, the kaposis fibroblast growth factor is present at 1 ng/ml to 50 ng/ml. In yet other embodiments, the nicotinamide is present at 1 mM to 50 mM. In some embodiments, the matrix is cleaved with a protease before the step of administering. In some embodiments, the protease is streptokinase or tissue plasminogen activator. In some embodiments, administration is by implantation under a kidney capsule of the mammal, subcutaneous, intravenous, via a liver portal vein, or into the pancreatic parenchyma. In some embodiments, the mammal is a human. In other embodiments, the islet cells are human islet cells. In yet other embodiments, the islet cells are autologous or heterologous.

Yet another embodiment of the present invention provides composition comprising isolated cells on a fibrin matrix. In some embodiments, the cells are islet cells or human islet cells. In some embodiments, the matrix is formed by mixing fibrinogen and thrombin. In other embodiments, the composition further comprises hepatocyte growth factor, kaposis fibroblast growth factor; or nicotinamide. In some embodiments, the hepatocyte growth factor is present from 10 ng/ml to 50 ng/ml. In other embodiments, the kaposis fibroblast growth factor is present at 1 ng/ml to 50 ng/ml. In even other embodiments, the nicotinamide is present at 1 mM to 50 mM.

Other embodiments and advantages of the present invention will be apparent from the detailed description that follows.

### BRIEF DESCRIPTION OF THE DRAWINGS

FIG. 1 illustrates a representative experiment of quantitation of growth (A-B), insulin content (C-D), and β-cell function (E) of islets cultured for 6 days free floating or in fibrin gels with or without the growth factor cocktail. For each parameter tested n=4; * p<0.05, ** p<0.005, *** p<0.0001. FIG. 1 shows that fibrin increases both total DNA and insulin content. FIG. 1 also shows that proliferation is induced with the addition of growth factors. Finally, FIG. 1 shows that fibrin does not impair the ability of β cells to respond to a glucose challenge.

FIG. 2 illustrates a representative short term adhesion assay showing inhibition of adhesion to fibrin substrates by function-blocking antibodies specific for αv, α5, and β1 integrin subunits but not by antibodies specific for αvβ3. The specific antibodies used were mAb L1A3 (specific for the av subunit), mAb P4C10 (specific for the β1 subunit), mAB LM609 (specific for the αvβ3 heterodimer) and mAb P1D6 (specific for the α5β1 heterodimer). For each parameter tested n=3; ** p<0.05; *** p<0.001.

### DETAILED DESCRIPTION OF THE INVENTION

I. Introduction

The present invention provides a method of culturing cells on a matrix comprising an α5β1 integrin, an αvβ1 ligand, or a combination thereof. For example, islet cells can be cultured in a 3-D configuration comprised of a fibrin matrix support. In addition, β-cell proliferation can be induced with HGF/SF. The methods of the present invention augment β-cell mass while preserving physiologic glucose responsiveness, i.e., insulin expression both in vitro and in vivo. Moreover, islet cells cultured according to the methods of the present invention do not de-differentiate and do not undergo senescence.

II. Definitions

As used herein, the following terms have the meanings ascribed to them below unless otherwise specified.

“Culturing” as used herein refers to maintaining cells under conditions in which they can proliferate, retain their differentiated state, and avoid senescence. For example, in the present invention, cultured islet cells proliferate and retain their insulin producing capacity. Cells can be cultured in growth media containing appropriate growth factors, i.e., a growth factor cocktail.

A “matrix” refers to a three dimensional support on which cells may be cultured. Cultured cells may be directly in contact with the matrix or the matrix may be linked with an α5β1 integrin ligand, an αvβ1 ligand, or a combination thereof before being contacted with the cells. A matrix is not a monolayer. A matrix may be any protein based or synthetic composition that forms a three dimensional support on which cells can be cultured.

An “α5β1 integrin ligand” is any compound that comprises a ligand for an α5β1 integrin receptor. The ligand may be a whole protein or a polypeptide fragment thereof. Suitable α5β1 integrin ligands include, for example, fibrin, fibronectin, and vitronectin. The α5β1 integrin ligand may be naturally occurring or recombinant.

An “αvβ1 integrin ligand” is any compound that comprises a ligand for an αvβ1 integrin receptor. The ligand may be a whole protein or a polypeptide fragment thereof. Suitable αvβ1 integrin ligands include, for example, fibrin, fibronectin, and vitronectin. The αvβ1 integrin ligand may be naturally occurring or recombinant.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission.

“Fibrin matrix,” “fibrin gel,” and “fibrin gel matrix” are used interchangeably herein to refer to a matrix formed by mixing fibrinogen and thrombin.

“Islet cells” refers to cells derived from the adult pancreatic tissue, fetal pancreatic tissue and islet-like cell clusters (ICCs). Islet cells may be α-cells. Islet cells preferably produce insulin. Islet cells can be from any type of mammal, e.g., a human, a cow, a pig, a sheep, a dog, a cat, a rat, or a mouse.

“Differentiation,” as used herein, refers to development of cells from primary cultures. For example, undifferentiated β cells or epithelial cells differentiate into insulin producing β cells.

“De-differentiation,” as used herein, refers to the loss of a cell's ability to perform its physiological function. For example, de-differentiation of β cells refers to a loss of their ability to produce insulin.

“Treating” or “treatment” refers to providing a therapeutically effective amount of cultured cells to a subject. A “therapeutically effective amount” of cells is an amount of cells that is sufficient to provide a therapeutic effect in a subject.

“Cleaving,” as used herein, refers to contacting a fibrin matrix which contains cultured cells with an amount of a protease that is sufficient to disrupt the matrix and dissociate the cells from the matrix. Suitable proteases include, for example, streptokinase or tissue plasminogen activator.

“Autologous,” when used in reference to cells, refers to cells obtained from the individual to whom the cells will be administered after being cultured according to the methods of the present invention.

“Heterologous,” when used in reference to cells, refers to cells obtained from a different individual from the individual to whom the cells will be administered after being cultured according to the methods of the present invention.

III. Culture Conditions

A. Matrices

Matrices useful for culturing cells according to the methods of the present invention comprise α5β1 integrin ligands, αvβ1 ligands, or a combination thereof. Suitable matrices include protein-based matrices such as, for example, fibrin or fibronectin, or synthetic matrices.

An exemplary protein based matrix is a fibrin clot. Such clots can be formed by mixing fibrinogen and thrombin in amounts suitable for forming a clot. For example, a solution comprising thrombin at about 20-100 U/ml may be mixed with a solution comprising fibrinogen at about 40-100 mg/ml. Generally, a solution comprising thrombin at about 50 U/ml is mixed with a solution comprising fibrinogen at about 80 mg/ml. In some embodiments, the fibrinogen and thrombin are mixed in a 1:1 ratio. Typically 2 mg fibrinogen are mixed with 1 U of thrombin. Fibrinogen and thrombin can be prepared using any means known in the art. For example, fibrinogen and thrombin may be naturally occurring or recombinant. Means of preparing fibrinogen complex from citrated plasma are taught, for example, in U.S. Pat. Nos. 4,650,678, 5,773,033, and 6,277,961 B1. A method of producing fibrinogen in transgenic mammals is disclosed in U.S. Pat. No. 5,639,940. A method of preparing fibrinogen from autologous plasma is disclosed in U.S. Pat. No. 5,773,033. EP 0103196 describes preparation of a fibrinogen concentrate from cryoprecipitate which, after thawing and dilution, has been treated with 2.5% Al(OH)₃. A method of preparing thrombin from lung tissue is described in U.S. Pat. No. 5,525,498. Method of preparing thrombin from mixtures comprising prothrombin are disclosed in U.S. Pat. Nos. 5,907,032; 5,945,103; and 6,168,938. U.S. Pat. No. 5,500,412 describes methods of preparing thrombin polypeptides. U.S. Pat. Nos. 5,527,692 and 5,502,034 disclose methods of producing recombinant thrombin.

Fibrin matrices comprising cells may be formed, for example, by adding cells to a solution comprising fibrinogen, then adding a solution comprising thrombin to the fibrinogen/cell mixture. One of skill in the art will understand that the order of addition of the matrix components is not a critical part of the present invention. Each component may be added simultaneously or sequentially in any order in accordance with the methods of the present invention.

In some embodiments, α5β1 integrin ligands, αvβ1 ligands, or a combination thereof, are presented on a synthetic matrix. Suitable synthetic matrices are described in, e.g., U.S. Pat. Nos. 5,041,138 and 5,512,474. For example, biodegradable artificial polymers, such as polyglycolic acid, polyorthoester, or polyanhydride may be used. When synthetic matrices are used, α5β1 integrin ligands, αvβ1 ligands, or a combination thereof are linked to the matrices. For example, fibronectin or vitronectin may be linked to an artificial polymer matrix to form a matrix comprising α5β1 integrin ligands, αvβ1 ligands, or a combination thereof. The fibronectin or vitronectin may be naturally occurring or recombinant. Methods of preparing vitronectin from plasma are described in, e.g., Barnes and Silnutzer (1983) J. Biol. Chem. 258(20):12548 and Dahlback and Podack (1985) Biochem. 24(9):2368. The linker may be introduced through recombinant means or chemical means. Suitable linkers include, for example, polypeptides that facilitate attachment of the fibronectin to the synthetic matrix. Methods of introducing linkers recombinantly are well known to those of skill in the art and are described in, e.g., Sambrook et al., Molecular Cloning, A Laboratory Manual (3d ed. 2001) and Current Protocols in Molecular Biology (Ausubel et al., eds., 1994)). Exemplary chemical linkages include, for example, covalent bonding, including disulfide bonding; hydrogen bonding; electrostatic bonding; recombinant fusion; and conformational bonding, e.g., antibody-antigen, and biotin-avidin associations. Additional linkers and methods of linking are described in WO 98/41641.

B. Cells

Any cell with α5β1 receptors, αvβ1 receptors, or a combination thereof may be cultured according to the methods of the present invention. Suitable cells include, for example, islet cells, other epithelial cells, endothelial cells, and retinal cells. Islet cells may be derived from, for example, adult pancreatic tissue, fetal pancreatic tissue and islet-like cell clusters (ICCs). Specifically, islet cells may be derived from the islets of Langerhans. Islets of Langerhans are clusters of cells in the pancreas that include the insulin-secreting β cells. Fetal pancreatic tissue is rich in undifferentiated β-cells that can grow and mature after transplantation. See, e.g., Tuch et al., (1986) Diabetes 35:464. ICCs are heterogeneous cell populations that include epithelial cells that differentiate after transplantation to form various types of cells including mature islets.

The cells to be cultured may be derived from any suitable mammal. For example the cells may be obtained from a rodents such as, for example, mice, rats, guinea pigs, and rabbits; non-rodent mammals such as, for example, dogs, cats, pigs, sheep, horses, cows, and goats; primates such as, for example, chimpanzees and humans. Suitable cells include pancreatic islets from adult humans or from the fetal pancreas. The cells to be cultured may be primary cells or may be cells maintained in culture. Techniques and methods for establishing a primary culture of cells for use in the methods of the invention are known to those of skill in the art. See e.g., Humason, ANIMAL TISSUE TECHNIQUES, 4^{th} ed., W. H. Freeman and Company (1979), and Ricciardelli et al., (1989) In Vitro Cell Dev. Biol. 25: 1016.

C. Growth Factors

Islet cells can be cultured in a growth factor cocktail comprising any or all of the following: hepatocyte growth factor (HGF), kaposis fibroblast growth factor (KFGF), and nicotinamide. Growth factors may be selected based on their ability to induce proliferation in the cells. Growth factors may also be selected based on their ability to prevent de-differentiation and senescence in the cells.

HGF is a 87 kDa two-chain glycoprotein cytokine first identified in rodent and human plasma and rodent blood platelets and is a potent hepatocyte mitogen. See, e.g., Rubin et al., (1993) Biochem. Biophys. Acta 1155:357, Miyazawa et al. (1989) Biochem. Biophys. Res. Commun. 163:967; Rubin et al., (1991) Proc. Nat'l Acad. (USA) 88:415; Weidner et al., (1991) Proc. Nat'l Acad. (USA) 88:7001; Nakamura et al., (1987) FEBS Lett. 224:311; Nakamura et al., (1989) Nature 342:440; Gohda et al., (1988) J. Clin. Invest. 81:414. HGF is also known as the fibroblast secretory protein, Scatter Factor (“SF”) which dissociates and increases the motility of epithelial cells. See, e.g., Gherardi et al. (1990) Nature 346:228; Weidner et al. (1991); Furlong et al. (1991) J. Cell Sci. 100:173; Naldini et al., EMBO J. 10:2867 (1991); Bhargava et al., Cell Growth Differ. 3:11 (1992). For reviews of HGF/SF, see Strain (1993) J. Endocrinol. 137:1, Furlong (1992) BioEssays 14:613, and Rubin et al. (1993). Typically, HGF is present in the growth factor cocktail at about 5 ng/ml to about 75 ng/ml, at about 10 ng/ml to about 50 ng/ml, or at about 20 ng/ml.

Typically, the heparin binding growth factor KFGF is present in the growth factor cocktail at about 0.5 to about 50 ng/ml, at about 0.75 to about 3.5 ng/ml, or at about 1 ng/ml.

Typically, the vitamin B3 derivative, nicotinamide, is also present in the growth factor cocktail. Generally, nicotinamide is present in the growth factor cocktail at about 1 mM to about 50 mM, at about 2 mM to about 40 mM, at about 5 mM to about 30 mM, or at about 10 mM.

Detection of the proliferation of islet cells may be accomplished by a variety of known techniques. For example, islet cell proliferation can also be detected by measuring the rate of DNA synthesis. Islet cells which have been stimulated to proliferate exhibit an increased rate of DNA synthesis. A typical way to measure the rate of DNA synthesis is, for example, by pulse-labeling cultures of islet cells with tritiated thymidine, a nucleoside precursor which is incorporated into newly synthesized DNA. The amount of tritiated thymidine incorporated can be determined using a liquid scintillation spectrophotometer. Other ways to detect islet cell proliferation include measuring production of insulin, production of the insulin by-product, C peptide (one molecule of C peptide is produced for each molecule of insulin), or dyes, such as 3-(4,5-dimethylthiazol-2-yl)-2,5-diphenyltetrazolium.

Techniques for detecting insulin or C-peptide include, for example, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al., J. Biol. Chem. 255:4980-4983 (1980)); radioimmunoassays (RIA); enzyme-linked immunosorbent assays (ELISA) as described, for example, by Raines et al., J. Biol. Chem. 257:5154-5160 (1982); immunocytochemical techniques, including the use of fluorochromes (Brooks et al., Clin. Exp. Immunol. 39:477 (1980)); and neutralization of activity (Bowen-Pope et al., Proc. Natl. Acad. Sci. USA 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

The differentiated state of cells can be detected by measuring the rate of insulin production or C peptide production. The differentiated state of cells can also be detected by analyzing the cell surface markers on the cells. For example, differentiated islet β-cells express PDX-1, but not CK-19 (see, e.g., Beattie et al. (1999) Diabetes 48:1013). Techniques for detecting cell surface markers are well known in the art and are described in, e.g., Harlow and Lane, USING ANTIBODIES (1999).

The differentiated state of cells can also be detected by analyzing the expression levels of various proteins by the cell. Methods of detecting protein expression are well known in the art and are described in, e.g., Ausubel et al., supra.

D. General Culturing Methods

This invention relies upon routine techniques in the field of cell culture. Suitable cell culture methods and conditions can be determined by those of skill in the art using known methodology (see, e.g., Freshney et al., CULTURE OF ANIMAL CELLS (3rd ed. 1994)). In general, the cell culture environment includes consideration of such factors as the substrate for cell growth, cell density and cell contract, the gas phase, the medium, and temperature.

Incubation is generally performed under conditions known to be optimal for cell growth. Such conditions may include for example a temperature of approximately 37° C. and a humidified atmosphere containing approximately 5% CO₂. The duration of the incubation can vary widely, depending on the desired results. In general, incubation is preferably continued until the cells begin to lose enough of their insulin secretion functionality to impose significant limits on their usefulness. As an approximate rule, the loss of over 60, 50, 40, 30, 25, 20, 15, or 10% of the rate of insulin secretion relative to fresh cells may be considered a limit. Proliferation is conveniently determined using ³H thymidine incorporation or BrdU labeling.

Plastic dishes, flasks, roller bottles, or microcarriers in suspension may be used to culture cells according to the methods of the present invention. Suitable culture vessels include, for example, multi-well plates, petri dishes, tissue culture tubes, flasks, roller bottles, and the like.

Cells are grown at optimal densities that are determined empirically based on the cell type. Cells are passaged when the cell density is above optimal.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions are used for cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media include DME, RPMI 1640, DMEM, Iscove's complete media, or McCoy's Medium (see, e.g., GibcoBRL/Life Technologies Catalogue and Reference Guide; Sigma Catalogue). Typically, RPMI 1640 is used in the methods of the invention. Defined cell culture media are often supplemented with 5-20% serum, typically heat inactivated serum, e.g., human, horse, calf, and fetal bovine serum. Typically, 10% fetal bovine serum is used in the methods of the invention. The culture medium is usually buffered to maintain the cells at a pH preferably from 7.2-7.4. Other supplements to the media include, e.g., antibiotics, amino acids, sugars, and growth factors. Typically, the media is supplemented with glucose at about 5.5-16.7 mM. In some embodiments, the glucose is present at about 11 mM.

IV. Administration of Cultured Cells

Cultured cells can be administered to a subject by any means known to those of skill in the art. Islet cells expanded, i.e., proliferated on a matrix, according to the methods of the present invention may facilitate 1:1 to 2:1 recipient:donor transplants to equal the success of whole organ pancreas transplants.

In one embodiment of the invention, cultured cells in an intact matrix may be administered to the subject. Alternatively, the matrix may be disrupted by a protease before the cultured cells are administered to the subject. Suitable proteases for disrupting the matrix include, for example, streptokinase or tissue plasminogen activator. Typically the protease is present at about a 2-4 fold excess relative to thrombin units used to make the clot.

In some embodiments of the present invention, islet cells are extracted from a human and subsequently contacted with a matrix comprising α5β1 integrin ligands, αvβ1 ligands, or a combination thereof. The embodiments are useful for treating diabetic subjects by implanting cells that express insulin in a glucose-dependent manner. Cells can be extracted from the subject to be treated, i.e., autologous, (thereby avoiding immune-based rejection of the implant) or can be from a second subject, i.e., heterologous. In either case, administration of cells can be combined with an appropriate immunosuppressive treatment.

Islet cells may be derived from, for example, adult pancreatic tissue, fetal pancreatic tissue and islet-like cell clusters (ICCs).

Cells cultured according to the methods of the present invention may be administered to a subject by any means known in the art. Suitable means of administration include, for example, intravenous, subcutaneous, via the liver portal vein, by implantation under the kidney capsule, or into the pancreatic parenchyma.

Suitable mammalian subjects include rodents such as, for example, mice, rats, guinea pigs, and rabbits, non-rodent mammals such as, for example, dogs, cats, pigs, sheep, horses, cows, and goats, primates such as for example, chimpanzees and humans.

Pharmaceutically acceptable carriers are determined in part by the particular composition being administered (e.g., cell), as well as by the particular method used to administer the composition. Accordingly, there are a wide variety of suitable formulations of pharmaceutical compositions of the present invention (see, e.g., Remington 's Pharmaceutical Sciences, 17^{th} ed., 1989).

The cells may be in formulations suitable for administration, such as, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by direct surgical transplantation under the kidney, intraportal administration, intravenous infusion, or intraperitoneal infusion.

Injection solutions and suspensions can be prepared from sterile powders, granules, and tablets. The dose administered to a patient, in the context of the present invention should be sufficient to effect a beneficial therapeutic response in the patient over time. The dose will be determined by the efficacy of the particular cells employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the cells to be administered in the treatment or prophylaxis of conditions owing to diminished or aberrant insulin expression, the physician evaluates cell toxicity, transplantation reactions, progression of the disease, and the production of anti-cell antibodies. For administration, cells of the present invention can be administered in an amount effective to provide normalized glucose responsive-insulin production and normalized glucose levels to the subject, taking into account the side-effects of the cell type at various concentrations, as applied to the mass and overall health of the patient. Administration can be accomplished via single or divided doses.

### EXAMPLES

The following examples are offered to illustrate, but not to limit the claimed invention.

### Example 1

### Materials and Methods

Human adult islets: Human adult islet preparations were provided through the JDFI Islet Distribution and the islet isolation facility at the University of California, San Diego. They were isolated with an automated method as described by Ricordi et al., (1988) Diabetes 37:413 and further purified by hand picking single islets after dithizone staining as described by Latif et al., (1988) Transplantation 45:827.

Islet cell culture: Islets were kept under 4 tissue culture conditions: free floating in RPMI containing 10% FBS and 11 mM glucose with or without a cocktail of growth factors, and in fibrin gel matrices also with or without the growth factor cocktail. The growth factor cocktail included 20 ng/ml recombinant human hepatocyte growth factor (HGF/SF; a generous gift from Genentech. Kaposis fibroblast growth factor (hrFGF4, 1 ng/ml; Oncogene Research Products), and 10 mM nicotinamide (Sigma, St. Louis, Mo.). Fibrin gel matrices were made by mixing human fibrinogen (Sigma, St. Louis, Mo.) dissolved in PBS (80 mg/ml) and human thrombin (Sigma, St. Louis, Mo.) dissolved in 40 mM CaCl₂ (50 U/ml). After placing the islets in 5 μl droplets of the fibrinogen an equal volume of the thrombin solution was immediately added to each drop. The liquid polymerized in about 12 minutes to a soft gel. At that time medium was added carefully to the dish. Medium and factors were replenished every 48 hours.

In vitro cellular growth and survival: Islets in each group were cultured for a total of 6 days. On day 5, after an overnight incubation in RPMI with the glucose concentration reduced to 5 mM, each group was tested for insulin release in response to glucose by incubating at 1.6 mM and 16.7 mM glucose concentrations. On day 6 proliferation rate was assayed by ³H thymidine incorporation as described by Otonkoski et al. (1993) J. Clin. Invest. 92:1459. DNA was measured by a fluorometric technique as described by Hinegardner (1971) AnalBiochem. 39:197. Insulin content and release were measured using a coated antibody RIA kit (DPC Los Angeles, Calif.) as described by Otonkoski et al. (1993).

Adhesion assays: Adhesion assays to determine specific integrin involvement were performed as described by Felding-Habermann et al. (1997) J. Cell Biol. 139:1567, with some modifications. Briefly, fibrinogen (20 μg/ml in PBS) was converted into fibrin in microtiter plates by the addition of 0.3 units/well thrombin solution (Sigma Corp), incubated at 37° C. for 1 hour followed by a blocking step for 1 hour using PBS, 5% BSA (Calbiochem). A single cell suspension of islet cells was washed several times in adhesion buffer (HBSS, 10 mM HEPES, 0.5% BSA, 1 mM CaCl₂, 1 mM MgCl₂ and 0.4 mM MnCl₂, pH 7.4). Cells were incubated with 50 μg/ml function-blocking anti-integrin antibodies specific for α3β1 (LM609) (Chemicon Temucula, Calif.), anti-α5β1 (P1D6) (Chemicon Temucula, Calif.) or β1 (P4C10) (Invitrogen Carlsbad, Calif.), then added to the wells and incubated at 37° C. until initial attachment was observed (at 90 minutes). Non-adherent cells were removed and adherent cells were fixed with 2% PFA. Adherent cells per microscope field were counted using an inverted stereomicroscope at 10×.

In vivo growth and survival: 500 islets cultured alone, or with fibrin and growth factors for 1 week were transplanted under the kidney capsule of athymic nude mice with a positive pressure pipet as described by Hayek and Beattie (1997) J. Clin. Endocrin. Metab. 82(8):2471, under anesthesia (phenobarbital i.p. 50 mg/kg). The fibrin gel matrices were dissolved 24 hours before transplantation with 3 U streptokinase/U thrombin (Sigma, St. Louis, Mo.). One month later, fasted animals were challenged with 3 g/kg glucose, and after 30 minutes blood samples were taken for assay of circulating human C-peptide, using a RIA assay kit that is specific for human C-peptide, with no cross reaction from mouse C-peptide (DPC Los Angeles, Calif.).

Immunohistochemical analysis in vitro: In some cultures, 1 mM BrdU was added to the cultures for 24-72 hours before harvesting. Islets were fixed in 4% paraformaldehyde, and embedded in paraffin. Sections were stained for insulin, BrdU and CK-19.

Immunohistochemical analysis in vivo: Grafts were removed and fixed in 4% paraformaldehyde, and embedded in paraffin. Sections were stained for insulin, glucagon and CK-19.

Antibodies used for both in vitro and in vivo immunohistochemical analysis were sheep anti-human insulin (The Binding Site), rabbit anti-human glucagon (Chemicon, Temucula, Calif.), mouse anti human cytokeratin 4.62 (CK-19) (Sigma. St. Louis, Mo.), and mouse anti BrdU (DAKO, Carpinteria, Calif.). Secondary Antibodies used were Rhodamine red-conjugated donkey anti-sheep, Fluorescein Isothiocyanate (FITC)-conjugated donkey anti-rabbit, or Indo-dicarbocyanine (CY5)-conjugated donkey anti-mouse IgGs (Jackson Immunoresearch). Control slides were incubated with a cocktail of the relevant control antibodies (mouse, sheep and/or rabbit IgGs).

Statistical analysis: Each experimental condition was tested on at least 6 different preparations of adult islets. Individual in vitro experiments used 4 replicate cell cultures for each parameter tested. Statistical significance of observed differences was analyzed by ANOVA and Fischer's protected least significance difference test with 95% level as the limit of significance, or student's T test of paired differences using Statview IV (Abacus Concepts, Berkeley, Calif.).

### Example 2

### Microscopic Analysis

A few hours after plating in fibrin gels, islets appeared slightly flattened by phase contrast microscopy. After 6 days, immunohistochemical staining showed that the majority of the cells in the fibrin gel were insulin positive. CK19⁺ cells were seen only rarely. BrdU labeling was observed only in the islets that were in fibrin gels and exposed to growth factors. BrdU⁺ cells were scattered throughout, but not in brightly staining insulin positive cells.

### Example 3

### Fibrin Promotes Survival and Growth

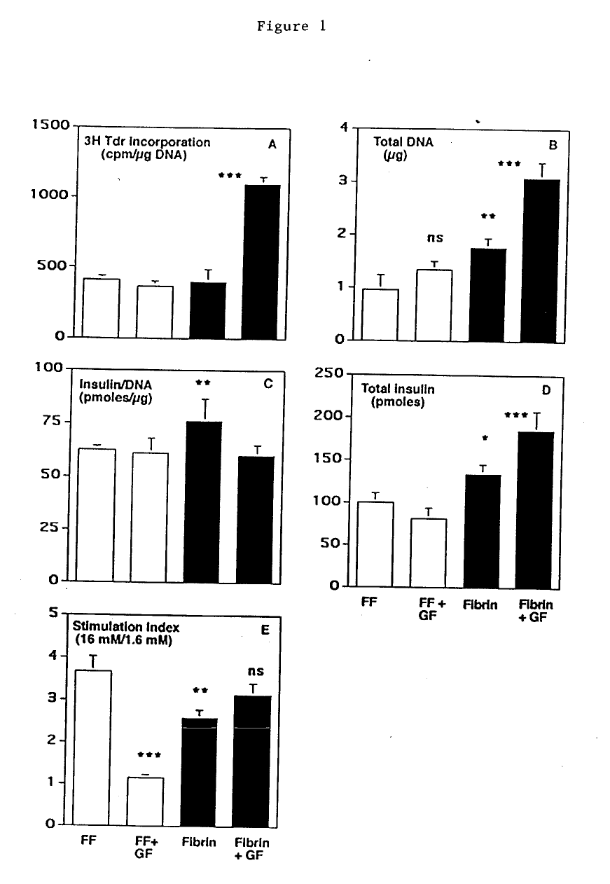

BrdU labeling data were validated by quantitative assays for thymidine incorporation. After 6 days in culture, proliferation was only observed in islets cultured in fibrin gels in the presence of the growth factor cocktail (FIG. 1A; n=4; p<0.0001); in this group the total DNA also increased 3 fold (FIG. 1B; n=4, p<0.0001). The islet cells were unable to proliferate in the presence of the growth factor cocktail alone. Although no proliferation was observed in the islets in fibrin without growth factors, as determined both by BrdU and ³H Tdr incorporation (FIG. 1A), there was a significantly higher total DNA, compared to free-floating islets, indicative of increased cell survival and suppression of cell death (FIG. 1B n=4; p<0.005). These data demonstrate that fibrin promotes survival. With the addition of growth factors, fibrin promotes proliferation of islet cells cultured in 3D configuration.

### Example 4

### Fibrin Promotes Increased β-Cell Mass

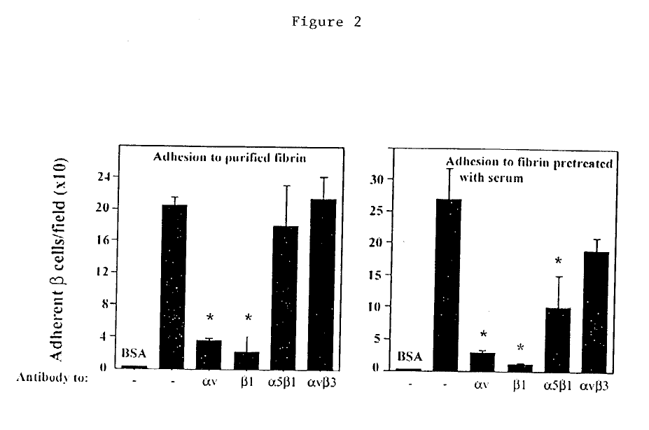

Both insulin/DNA and total insulin were increased significantly after 6 days in fibrin gels (FIG. 1C; n=4, p<0.005, FIG. 2D; n=4 p<0.05). Thus the addition of fibrin alone is sufficient to increase β-cell mass. When the growth factors were added, the total insulin increased 2 fold (FIG. 1D; n=4 p<0.0001) indicating a doubling in β-cell mass. These findings differ significantly from the situation when cells are grown in monolayer in the presence of growth factors with a concomitant loss of insulin expression (see, e.g., Beattie et al. (1999) Diabetes 48:1013).

The phenotype of maximally expanded endocrine cells under the influence of matrix and HFGF/SF is PDX-1 positive (see, e.g., Beattie et al (1999) Diabetes 48:1013), Beta2/NeuroD negative. We also determined that FGF-4 also helps maintain the β-cell phenotype in HGF/SF expanded islet cells. Our findings in this report show that while growth factors are ineffective in islets that are free floating, cellular interaction in a three dimensional configuration with a fibrin matrix allows proliferation to occur. Thus monolayers are not necessary for proliferation. By not culturing the cells in a monolayer, there is no need to disrupt cell matrix interactions. Thus, the anoikis, i.e., binding to the extracellular matrix through inappropriate integrins, that can be associated with disruption of cell matrix interactions can be avoided.

### Example 5

### Islets are Still Functional in Fibrin Gels

After 5 days in culture, all groups, except for the islets that were free floating in the presence of the growth factor cocktail, were capable of being stimulated at least 2 fold with a glucose challenge (FIG. 1E). These results show that #-cell function is not impaired in proliferating islet cells ligated to fibrin, in contrast to the loss of function seen previously in islets expanded in monolayer on matrices such as HTB-9.

### Example 6

### Integrin α5β1, αvβ1, or Combinations Thereof Support β-Cell-Fibrin Interaction

To identify integrins responsible for β-cell-fibrin interaction, short term adhesion assays were performed in the presence of function blocking antibodies to the αv subunit, the β1 subunit or to specific integrin heterodimers, including αᵥβ₃ and α5β1. Importantly blockade of the α5β1 heterodimer, the β1 subunit, or the av subunit resulted in a significant inhibition of adhesion to fibrin substrates (FIG. 2). Fibrinogen preparations are commonly contaminated with fibronectin; a recognized ligand for α5β1 (see, e.g., Piotrowicz et al. (1988) J. Cell Biol. 106:1359). However we demonstrated that adhesion assays using a high purity fibrinogen preparation free of fibronectin still resulted in α5β1-dependent adhesion.

We found that the efficiency of adhesion to fibrin was inversely related to time in culture. Our results also demonstrate that ex vivo expansion of β-cells in the fibrin matrices is enhanced with the incorporation of freshly isolated islet cell preparations into the fibrin gels.

### Example 7

### Islet Engraftment is Improved After Culture in Fibrin Gels

Macroscopically, grafts appeared larger in mice that had been transplanted with islets grown in fibrin gels compared to islets cultured free floating. This observation was confirmed by immunostaining, which showed large compact grafts of endocrine cells. Most of the cells stained for insulin or glucagon; CK19⁺ cells were rarely seen. In contrast, grafts from free-floating islets were smaller and showed scattered endocrine cells and also some ductal structures.

After glucose challenge of fasted mice, circulating human C-peptide was significantly 2 fold higher in mice receiving the graft from fibrin treated islets than in the mice receiving a comparable number of free-floating islets from the same islet preparation (Table 1; n=6; p<0.005).

The results show that previous culture in fibrin gels ensures a robust graft, capable of withstanding the deleterious conditions of engraftment. Dissolution of the gel by substances such as streptokinase (e.g., 2-4 Units per unit thrombin) or tissue plasminogen activator would facilitate the human use of islets so treated. Limited ex vivo islet expansion using fibrin and growth factors may facilitate 1:1 recipient:donor transplants to equal the success of whole organ pancreas transplants.

Fibrin promotes survival, and allows for HGF-mediated cell proliferation while preserving glucose responsiveness by cell-to-cell contacts. Thus preservation of function in vitro requires 3-D configuration provided by a fibrin or other α5β1 ligand matrix support.

### Example 8

### Comparison of Human C-Peptide Levels

Mice were transplanted with islets previously cultured under free floating conditions or in a fibrin matrix. One month after transplantation, mice were fasted overnight, then challenged with glucose i.p. (3 g/kg). Thirty minutes after the glucose challenge, blood was drawn from the external jugular and assayed for serum levels of human C-peptide using and RIA kit (DPC, Los Angeles, Calif.).

The results are expressed as pmol C-peptide per liter in Table 1 below.

TABLE 1

| Experiment # | Control Grafts | Fibrin Grafts | Increase |
|---|---|---|---|
| 1 | 184 | 457 | 2.5 |
| 2 | 147 | 588 | 4 |
| 3 | 327 | 648 | 2.0 |
| 4 | 204 | 347 | 1.7 |
| 5 | 938 | 1644 | 1.8 |
| 6 | 674 | 1134 | 1.7 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of culturing cells, the method comprising:
culturing the cells on a matrix, wherein the matrix comprises an integrin ligand, wherein the integrin ligand is selected from the group comprising an α5β1 integrin ligand and an αvβ1 integrin ligand.

2. The method according to claim 1, wherein the integrin ligand is an α5β1 integrin ligand.

3. The method according to claim 1, wherein the integrin ligand is an α\overline{ω}β1 integrin ligand

4. The method according to claim 1, wherein the matrix further comprises a synthetic mesh.

5. The method according to claim 1, wherein the integrin ligand is fibrin.

6. The method according to claim 1, wherein the integrin ligand is fibronectin.

7. The method according to claim 1, wherein the integrin ligand is vitronectin.

8. The method according to claim 1, wherein the culturing step comprises mixing fibrinogen and thrombin, thereby forming a matrix comprising fibrin.

9. The method according to claim 1, wherein the cells are islet cells.

10. The method according to claim 9, wherein the cells are human islet cells.

11. The method according to claim 1, wherein the method further comprises contacting the cells with hepatocyte growth factor.

12. The method according to claim 11, wherein the culturing step further comprises contacting the cells with:
kaposis fibroblast growth factor; and
nicotinamide.

13. The method according to claim 12, wherein the human hepatocyte growth factor is present at 20 ng/ml;
the kaposis fibroblast growth factor is present at 1 ng/ml; and
the nicotinamide is present at 10 mM.

14. The method according to claim 1, whereby dc-differentiation of the cells is prevented.

15. The method according to claim 1, whereby senescence of the cells is prevented.

16. A method of cell transplantation, the method comprising the steps of:
culturing cells on a matrix, wherein the matrix comprises an integrin ligand, wherein the integrin ligand is selected from the group comprising an α5β1 integrin ligand and an αvβ1 integrin ligand; and
administering the cells to a mammal.

17. The method according to claim 16, wherein the integrin ligand is a α5β1 integrin ligand.

18. The method according to claim 16, wherein the integrin ligand is a αvβ1 integrin ligand.

19. The method according to claim 16, wherein the cells are administered to a diabetic animal.

20. The method according to claim 16, wherein the cells are islet cells.

21. The method according to claim 16, wherein the matrix further comprises a synthetic mesh.

22. The method according to claim 16, wherein the integrin ligand is fibrin.

23. The method according to claim 16, wherein the integrin ligand is fibronectin.

24. The method according to claim 16, wherein the integrin ligand is vitronectin.

25. The method according to claim 16, wherein the culturing step comprises mixing fibrinogen and thrombin, thereby forming a matrix comprising fibrin.

26. The method according to claim 16, wherein the culturing step comprises contacting the cells with hepatocyte growth factor.

27. The method according to claim 26, wherein the culturing step further comprises contacting the cells with:
kaposis fibroblast growth factor; and
nicotinamide.

28. The method according to claim 27, wherein the human hepatocyte growth factor is present at 20 ng/ml;
the kaposis fibroblast growth factor is present at 1 ng/ml; and
the nicotinamide is present at 10 mM.

29. The method according to claim 16, further comprising cleaving the matrix with a protease before the step of administering.

30. The method according to claim 29, wherein the protease is streptokinase.

31. The method according to claim 16, wherein the administration is by implantation under a kidney capsule of the mammal.

32. The method according to claim 16, wherein the administration is subcutaneous.

33. The method according to claim 16, wherein the administration is intravenous.

34. The method according to claim 16, wherein the administration is via a liver portal vein.

35. The method according to claim 16, wherein the mammal is a human.

36. The method according to claim 16, wherein the islet cells are human islet cells.

37. The method according to claim 16, wherein the islet cells are autologous.

38. The method according to claim 16, wherein the islet cells are heterologous.

39. A composition comprising isolated cells on a fibrin matrix.

40. The method according to claim 39, wherein the cells are islet cells.

41. The method according to claim 40, wherein the cells are human islet cells.

42. The composition according to claim 39, wherein the matrix is formed by mixing fibrinogen and thrombin.

43. The composition according to claim 39, further comprising hepatocyte growth factor.

44. The composition according to claim 43, further comprising:
kaposis fibroblast growth factor; and
nicotinamide.

45. The composition according to claim 44, wherein the human hepatocyte growth factor is 20 ng/ml;
the kaposis fibroblast growth factor is 1 ng/ml; and
the nicotinamide is 10 mM.
